(19) United States
(12) Patent Application Publication
(10) Pub. No.: US 2023/0046381 A1
(43) Pub. Date: Feb. 16, 2023

(54) NUCLEIC ACID AND CELL PRESERVATIVE COMPOSITIONS AND METHODS OF USE

(71) Applicant: SIO2 Medical Products, Inc., Auburn, AL (US)

(72) Inventors: Joseph Piccirilli, Chicago, IL (US); Christopher Weikart, Auburn, AL (US); Alexander M. Klibanov, Auburn, AL (US)

(21) Appl. No.: 17/791,722

(22) PCT Filed: Jan. 8, 2021
(86) PCT No.: PCT/US2021/012844

§ 371 Date: Jul. 8, 2022

Related U.S. Application Data
(60) Provisional application No. 63/011,468, filed on Apr. 17, 2020.
(60) Provisional application No. 62/959,818, filed on Jan. 10, 2020.

(51) Int. Cl. C12Q 1/6806; C12N 15/10

(52) U.S. Cl. CPC C12Q 1/6806; C12N 15/1003

(57) ABSTRACT

Nucleic acid and cell preservative compositions. Methods of preserving nucleic acids and/or cells in a blood or other biological sample, and kits for preserving nucleic acids and/or cells in a blood or other biological sample are also described. The preservative compositions, methods and kits afford the isolation of genomic and cell-free DNA and RNA from myriad biological samples that display good yields, purity, integrity and, in the context of RNA, amplifiability.

### BACKGROUND OF THE DISCLOSURE

A number of nucleic acid-based tests are used to analyze variations in the sequence, structure or expression of DNA and RNA for a variety of diagnostic purposes. Indeed, nucleic acids are common examination targets for non-invasive biomedical studies. However, after a biological sample has been collected, the nucleic acids within that sample, e.g., RNA and DNA, whether cellular/genomic or cell free, begin to degrade. Furthermore, gene induction and the degradation of gene transcripts begin occurring within minutes of blood or other biological sample collection making it difficult to accurately analyze the gene expression of the sample at the time it is collected. Moreover, in general, the fresher the blood or other biological sample, the better the quality of the nucleic acids of that sample will be. This presents a problem when the nucleic acids of a subject's blood or biological sample are to be analyzed. It is often the case that blood and other biological samples are collected at a different location and at a very different time than where and when they are analyzed. For this reason, after blood or other biological samples are collected, they need to be stored and transported before they can be analyzed. Due to the rapid degradation of nucleic acids that occurs in blood and other biological samples after they are collected, there is a need for a method to preserve the nucleic acids that are present in samples in order to ensure that the nucleic acids of the samples are of high quality at the time they are analyzed.

In the case of cell free nucleic acids in a blood or other biological sample, the different location and timing of collection and analysis present an additional problem: cell lysis. Cell lysis in the collected sample may lead to the contamination of the cell free nucleic acid profile with cellular nucleic acids, making it difficult to accurately analyze the cell free nucleic acids in the biological sample. Cell lysis begins to occur soon after blood or other biological samples have been collected. This presents a problem when the samples need to be stored for an extended period of time prior to being analyzed. Thus, there is a further need to preserve blood and other biological samples such that the cell free profile of its nucleic acids is maintained.

Similarly, for diagnostic applications that are based on the detection or analysis of cells, e.g., circulating tumor cells in a biological sample, the preservation of those cells in their intact form is important.

### SUMMARY OF THE DISCLOSURE

In one aspect, the disclosure is directed to a nucleic acid and cell preservative composition comprising:

a. one or more of an osmotic agent;

b. one or more of an enzyme inhibitor;

c. optionally one or more of a metabolic inhibitor;

d. optionally a plasma expander; and

e. one or more of an agent selected from a hydroxyethylstarch, a polymer of N-vinylpyrollidone (NVP), a Ficoll, a protein colloid, a non-protein synthetic colloid, ethylene diol, propylene glycol, a water-soluble polymer and carboxymethylcellulose or a salt thereof.

In another aspect, the disclosure is directed to a method for preserving nucleic acids in a biological sample comprising the steps of combining a preservative composition of the disclosure and the biological sample.

In one aspect, the disclosure is directed to a method for preserving cells in a biological sample comprising the steps of combining a preservative composition of the disclosure and the biological sample.

In a further aspect, the disclosure is directed to a kit for preserving nucleic acids and/or cells in a biological sample comprising:

a. a preservative composition disclosed herein; and

b. optionally, instructions for use of said preservative composition.

In another aspect, the disclosure is directed to a kit for preserving nucleic acids and/or cells in a biological sample comprising:

a. a blood or other biological sample collection tube optionally containing a predetermined amount of an optional anticoagulant;

b. a syringe containing a predetermined amount of a preservative composition disclosed herein; and

c. optionally, a needle attachable to said syringe.

In a further aspect, the disclosure is directed to a kit for preserving nucleic acids and/or cells in a biological sample comprising:

a. a blood or other biological sample collection tube optionally containing a predetermined amount of an anticoagulant; and

b. an ampule, containing a predetermined amount of a preservative disclosed herein, wherein said ampule comprises a removable closure and wherein said ampule is configured to receive a dispensing means upon removal of the closure by a user.

In another aspect, the disclosure is directed to a kit for preserving nucleic acids and/or cells in a biological sample comprising a collection tube optionally containing a predetermined amount of an anticoagulant and a preservative composition disclosed herein.

In one aspect of the disclosure, the nucleic acids are cell free (“cf”) DNA. In another aspect of the disclosure, the nucleic acids are cellular (i.e., genomic or “g”) DNA.

In one aspect of the disclosure, the nucleic acids are cell free (“cf”) RNA. In another aspect of the disclosure, the nucleic acids are cellular (i.e., genomic or “g”) RNA.

In another aspect of the disclosure, the cells are stem cells, bone cells, blood cells (e.g., red blood cells and/or white blood cells), muscle cells, fat cells, skin cells, nerve cells, endothelial cells, sex cells, pancreatic cells, cancer cells, tumor cells, or circulating tumor cells. In some embodiments, the cells are lab-derived or modified cells.

### DETAILED DESCRIPTION OF THE DISCLOSURE

### Definitions

Unless otherwise defined herein, scientific and technical terms used in this application shall have the meanings that are commonly understood by those of ordinary skill in the art. In case of conflict, the present specification, including definitions, will control.

Throughout this application and its various embodiments and aspects, the word “comprise,” or variations such as “comprises” or “comprising,” will be understood to allow the inclusion of a stated integer or group of integers but not the exclusion of any other integer or group of integers.

The term “including” or “includes” is used to mean “including but not limited to.” “Including” and “including but not limited to” are used interchangeably.

Any example(s) following the term “e.g.” or “for example” is not meant to be exhaustive or limiting to this disclosure.

Unless otherwise required by context, singular terms shall include pluralities and plural terms shall include the singular.

The articles “a”, “an” and “the” are used herein to refer to one or to more than one (i.e., to at least one) of the grammatical object of the article.

All ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10. The term “about” when used in the context of the weight percentage of a component means +/−10% of the recited number.

Each embodiment of this disclosure may be taken alone or in combination with one or more other embodiments of this disclosure.

Exemplary methods and materials are described herein, it should be understood that methods and materials similar or equivalent to those described herein can also be used in the practice or testing of the various aspects and embodiments. The materials, methods, and examples are illustrative only and not intended to be limiting.

In order for the disclosure to be more readily understood, certain terms are first defined. These definitions should be read in light of the remainder of the disclosure as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, the term “osmotic agent” refers to an agent that produces a hypertonic, and in some embodiments an isotonic, solution. Examples of osmotic agents include, but are not limited to, hypertonic or isotonic salt solutions, including, but not limited to solutions containing, for example, sodium, potassium, magnesium and calcium salts, Ringer's lactate, Ringer's acetate, an amino acid, sorbitol, glycerol, mannitol, sugars such as sucrose or glucose, tartaric acid, and glucaric acid or salts of any of them. Without wishing to be bound by theory, the one or more osmotic agents serve to increase osmotic pressure in the blood or other biological sample, leading to the release of water from the cells present in the blood or biological sample to counteract the imbalance. This causes the cells to shrink, thereby, making them more resistant to cell lysis which would otherwise cause the cell-free nucleic acids of the biological sample to be contaminated with cellular nucleic acids, or the cells to be less amenable to assay and analysis. Additionally, it is believed that the optional plasma expander will enhance this effect.

As used herein, the term “enzyme inhibitor” refers to an agent that, alone or in a preservative composition of this disclosure, generates complexes with metal ions, such as, for example calcium, magnesium, manganese or zinc, to reduce blood coagulation, inhibit nucleases and/or reduce enzymatic cell lysis. Examples of enzyme inhibitors of this disclosure include but are not limited to ethylenediaminetetraacetic acid (EDTA), hydroxyethylethylenediaminetriacetic acid (HEDTA), dithiothreitol (DTT), ethylene glycol-bis(β-aminoethyl ether)-N,N,N′,N′-tetraacetic acid (EGTA), citric acid, oxalate, aurintricarboxylic acid (ATA), tartaric acid, glucaric acid, or salts of any of them, including but not limited to sodium and potassium salts. Without wishing to be bound by theory, the inhibition of nucleases will prevent or reduce the degradation of cell-free nucleic acids within the biological sample. Examples of enzymes that the enzyme inhibitor of this disclosure inhibit, include, but are not limited to lysostaphin, zymolase, protease, glycanase, or other enzymes that are known to induce cell lysis, thereby acting to preserve the cells of blood or other biological or lab-derived samples.

As used herein, the term “metabolic inhibitor” refers to an agent that, alone or in a preservative composition of this disclosure, inhibits cellular processes, such as cellular respiration, cellular metabolism and metabolic function. The metabolic inhibitor of this disclosure is believed to slow the growth of cells by inhibiting cell metabolic functions and suppressing bacterial growth, thereby reducing degradation of cell-free nucleic acids. Examples of metabolic inhibitors of this disclosure include, but are not limited to, sodium azide, thimerosal, proclin, or chlorohexidine.

As used herein, the term “plasma expander” refers to an agent that produces a hyperoncotic or hypertonic solution. Examples of plasma expanders include, but are not limited to glycerol, starch, protein colloids (e.g., albumin, ovalbumin, and gelatins) and non-protein colloids (e.g., hydroxyethyl starch). Without wishing to be bound by theory, the one or more plasma expanders of the compositions of this disclosure also serve to increase osmotic pressure in the blood plasma or other biological sample, leading to the release of water from the cells to counteract the imbalance. This causes the cells to shrink, thereby, making them more resistant to cell lysis which would otherwise cause the cell-free nucleic acids of the biological sample to be contaminated with cellular nucleic acids, or the cells to be less amenable to assay and analysis.

As used herein, the term “hypertonic saline solution” refers to a salt solution with a salt concentration that is higher than physiologic. Examples of hypertonic saline solutions include, but are not limited to a 2%, 3%, 4%, 5%, 6%, 7%, 8%, 9%, 10%, 12%, 14%, 15%, 16%, 17%, 18%, 19%, 20%, 21%, 22%, 23%, 24% and 25% NaCl solution. Hypertonic saline solutions can also be characterized by salts, other than NaCl, including, but not limited to KCl, CaCl₂ and KNO₃.

As used herein, the term “isotonic saline solution” refers to a salt solution with a salt concentration that is equal to physiologic, thus there is no net water movement or change in the size of the cell. Examples of isotonic saline solutions include, but are not limited to a 0.5%, 0.7%, and 1% NaCl solution. Isotonic saline solutions can also be characterized by salts, other than NaCl, including, but not limited to KCl, CaCl₂ and KNO₃.

As used herein, a “Ficoll” refers to a water soluble high molecular weight sucrose polymer that is formed from the polymerization of sucrose with epichlorohydrin. For example, Ficoll 400 and Ficoll 70.

As used herein, a “protein colloid” refers to a mixture in which one or more proteins is dispersed in solution. Examples of protein colloids of this disclosure include, but are not limited to albumin, ovalbumin, or gelatins. The albumin may be provided as, for example, a human serum albumin (HAS), a bovine serum albumin (BSA) or an ovalbumin. Examples of gelatins include, but are not limited, to urea-linked gelatins (e.g., Haemaccel®), succinylated gelatins (e.g.,)Gelofusine®), and oxypolygelatins.

As used herein, the term “a non-protein colloid” refers to a mixture in which one or more large molecules or ultramicroscopic particles are dispersed in solution. Examples of non-protein colloids include, but are not limited to, branched natural polymers of amylopectin, such as hydroxyethylated starches (HES), and polysaccharides, such as dextrans, for example, Dextran 40 and/or Dextran 70.

As used herein, a “water-soluble polymer” refers to a polymer that is soluble in aqueous solution. Examples of water-soluble polymers includes, but are not limited to a polyacrylamide, a polyacrylate, a polydextrose, a polyglycine, a polyethyleneimine, a polylysine, a polyethylene glycol, a polyvinyl pyrrolidone, a polyvinyl alcohol, a polyacrylic acid, a polymer of N-(2-hydroxypropyl) methacrylamide, a polymer of divinyl ether-maleic anhydride, a polyoxazoline, a polyphosphate, a polyphosphazene, a xanthan gum, a pectin, a chitosan derivative, a dextran, a carrageenan, a guar gum, a cellulose ether, a sodium carboxymethyl cellulose, a hydroxypropyl cellulose, a hypromellose, a hyaluronic acid, an albumin, a starch, or a starch based derivative. For further non-limiting examples of water-soluble polymers of the disclosure, see Betageri, G.V., Kadajji, V. G., Polymers, 2011, 3, pp. 1972-2009.

As used herein, the term “nucleic acid” includes both ribonucleic acid (RNA) and deoxyribonucleic acid (DNA). The RNA and/or DNA may be linear or branched, single or double stranded or fragmented. The RNA and DNA may be cellular RNA (i.e., genomic RNA), cellular DNA (i.e., genomic DNA), cell-free RNA, cell-free DNA or combinations thereof. Nucleic acids are found in biological samples, and in particular, blood samples.

As used herein, the term “biological sample” refers to a sample obtained from a biological source, including lab-derived or lab-modified cells that comprises nucleic acids and/or cells. Biological samples may be cell, culture or tissue samples. Additionally, biological samples may be derived from bodily fluids, such as, for example, blood, plasma, serum, urine, saliva, stool, breast milk, tears, sweat, cerebral spinal fluid, synovial fluid, semen, vaginal fluid, ascitic fluid, amniotic fluid, or cell culture media.

As used herein, the term “preservative” refers to a composition that is added to a biological sample that inhibits, prevents, or slows the degradation of the nucleic acids and/or cell lysis in that sample.

As used herein, the term “treated biological sample” refers to a biological sample that has been combined with a preservative composition as described in this disclosure.

As used herein, the term “cells” refers to any cell that may be found in blood or other biological samples. Types of cells include, but are not limited to stem cells, bone cells, blood cells (e.g., red blood cells or white blood cells), muscle cells, fat cells, skin cells, nerve cells, endothelial cells, sex cells, pancreatic cells, cancer cells, tumor cells, circulating tumor cells (CTCs) and lab derived and/or modified cells.

### Preservative Compositions of the Disclosure

The compositions of this disclosure are useful in the preservation and stabilization of nucleic acids and/or cells in biological samples. When the preservative compositions of the disclosure are added to a biological sample containing nucleic acids and/or cells, the degradation of the nucleic acids and/or cell lysis in that sample is reduced, slowed or prevented, as compared to untreated biological samples, allowing for the subsequent isolation and more accurate analysis of the nucleic acids and/or the cells via conventional techniques known in the art. Additionally, the preservative compositions of the disclosure inhibit, slow, or reduce cell lysis, allowing the cell free nucleic acids in the sample to remain more consistent in amount and character over prolonged periods of time. The reduction of cell lysis in treated biological samples also reduces the release of nucleases, thereby further preventing or reducing degradation of nucleic acids and/or cells within the sample. The nucleic acids that can be preserved by the compositions of the disclosure include RNA, DNA or combinations thereof. The RNA and DNA can be cellular or cell-free or combinations thereof, i.e., cellular RNA, cellular DNA, cell-free RNA, cell-free DNA, or combinations thereof. Preferably, the DNA and/or RNA is cell-free DNA and/or RNA. The cells, whose lysis is reduced using the compositions and methods of this disclosure, can be, without limitation, stem cells, bone cells, blood cells, muscle cells, fat cells, skin cells, nerve cells, endothelial cells, sex cells, pancreatic cells, cancer cells, tumor cells, circulating tumor cells and lab-derived or modified cells.

In one aspect, the preservative composition of the disclosure comprises:

a. one or more of an osmotic agent;

b. one or more of an enzyme inhibitor;

c. optionally one or more of a metabolic inhibitor;

d. optionally a plasma expander; and

e. one or more of an agent selected from a hydroxyethylstarch, a polymer of N-vinylpyrollidone (NVP), a Ficoll, a protein colloid, a non-protein synthetic colloid, ethylene diol, propylene glycol, a water-soluble polymer and carboxymethylcellulose or a salt thereof.

Without wishing to be bound by theory, the one or more agents selected from a hydroxyethylstarch, a polymer of N-vinylpyrollidone (NVP), a Ficoll, a protein colloid, a non-protein synthetic colloid, ethylene diol, propylene glycol, a water-soluble polymer and carboxymethylcellulose or a salt thereof, serve as a crowding agent, forcing cells out of solution thereby preventing or reducing cell lysis and the subsequent degradation of the cells or release of cellular nucleic acids into the sample that may otherwise contaminate, for example, the cell-free nucleic acids within the sample. The nucleic acids and/or cells can subsequently be isolated and more accurately analyzed via conventional methods known in the art.

In some embodiments, the one or more agents are present in an amount of about 10% to about 50% by weight of the composition. For example, in some embodiments, the one or more agents are present in an amount of about 10% to about 40% by weight, or from about 15% to about 35% by weight, or from about 20% to about 30% by weight of the composition.

In some embodiments, the one or more agents are a protein colloid.

In some embodiments, the one or more agents are a non-protein colloid.

In some embodiments, the one or more agents are a water-soluble polymer.

In some embodiments, the osmotic agent(s) is present in the preservative compositions of the disclosure in an amount of about 0.5% to about 20% by weight of the composition. In some embodiments, the osmotic agent is present in an amount of about 1% to about 15%, about 1% to about 20% or about 0.5% to about 10% by weight of the composition.

In some embodiments, the one or more osmotic agents are hypertonic or isotonic salt solutions, including, but not limited to sodium, potassium, magnesium and calcium containing solutions, Ringer's lactate, Ringer's acetate, an amino acid, sorbitol, glycerol, mannitol, sugars such as sucrose or glucose, tartaric acid, or glucaric acid or salts of any of them.

In some embodiments, the enzyme inhibitor(s) is present in the preservative compositions of the disclosure in an amount of about 0.5% to about 30% by weight, in some aspects in an amount of about 0.5% to about 5% by weight and in other aspects from about 1% to about 30% by weight, of the composition. In some embodiments, the enzyme inhibitor(s) is present in an amount of about 1% to about 20% by weight of the composition. In another embodiment, the enzyme inhibitor is present in an amount of about 1% to about 10% by weight of the composition.

In some embodiments, the one or more enzyme inhibitors are ethylenediaminetetraacetic acid (EDTA), hydroxyethylethylenediaminetriacetic acid (HEDTA), dithiothreitol (DTT), ethylene glycol-bis(β-aminoethyl ether)-N,N,N′,N′-tetraacetic acid (EGTA), citric acid, oxalate, aurintricarboxylic acid (ATA), tartaric acid, glucaric acid, or salts of any of them. The salts included but are not limited to sodium and potassium salts or mixtures of them.

In some embodiments, the one or more optional metabolic inhibitors are present in the preservative compositions of the disclosure in an amount of about 0.01% to about 10% by weight of the composition. In another embodiment, the optional metabolic inhibitor is present in an amount of about 0.01% to about 5% by weight of the composition. In another embodiment, the optional metabolic inhibitor is present in an amount of about 0.01% to about 2% by weight of the composition.

In some embodiments, the one or more optional metabolic inhibitors are sodium azide, thimerosal, proclin or chlorohexidine.

In some embodiments, the optional plasma expander is present in the preservative compositions of the disclosure in an amount of about 0.5% to about 40% by weight of the composition, such as about 0.5%, about 1%, about 10%, about 15%, about 20%, about 25%, about 30%, about 35% or about 40% by weight of the composition.

In some embodiments, the optional plasma expander is glycerol, starch, protein colloids (e.g., albumin, ovalbumin, and gelatins) or non-protein colloids (e.g., hydroxyethyl starch).

In some embodiments, one or more components of the preservative composition of this disclosure may serve the role or function of one or more of the components of the preservative composition. For example, tartaric acid or glucaric acid or a salt thereof may be present in the compositions of the disclosure as an enzyme inhibitor, an osmotic agent, or both. As an additional example, human serum albumin may be present in the compositions of the disclosure as a one or more agent, an optional plasma expander, or both. As an additional example, glycerol may be present in the compositions of the disclosure as a one or more agent, an osmotic agent, a low molecular weight volume excluding agent, a plasma expander, or may provide some or all of these functions.

The preservative composition according to the disclosure can be in the form of a lyophilized powder, granules, tablets, or as a solution (e.g., wherein the preservative composition is reconstituted in a suitable vehicle). The lyophilized powder, granules and/or tablets may be added directly to the biological sample or may be reconstituted prior to being added to a biological sample. The lyophilized powder, granules, and/or tablets may, for example, be reconstituted by dissolving the composition in a suitable vehicle. Suitable vehicles include but are not limited to water, saline, Ringer's solution, fixed oils of vegetable origin, mono and diglycerides of fatty acids, ethanol, glycerin, propylene glycol, and an optional plasma expander of this disclosure. Alternatively, the biological sample may be added to the lyophilized powder, granules, tablets or the reconstituted composition (i.e., solution) directly. As another example, when the biological sample is derived from a bodily fluid, the bodily fluid can serve as an acceptable vehicle for solubilizing the preservative composition. For example, the lyophilized powder, granule, and/or tablet form of the preservative composition can be combined with the bodily fluid, thereby being solubilized by the bodily fluid. In some embodiments, the collection tube or container contains the preservative composition as a lyophilized powder, granule, tablet or solution before the biological sample is collected in the tube or container.

In some embodiments, the preservative composition of the disclosure is in the form of an aqueous solution. The aqueous solution may be combined with a biological sample or the biological sample combined with the aqueous solution.

Methods of Preserving Nucleic Acids and/or Cells of Biological Samples

In one aspect, the disclosure is directed to a method for preserving nucleic acids and/or cells in a biological sample comprising combining a preservative composition of this disclosure and the biological sample. In some embodiments, the biological sample is a cell or tissue sample. In some embodiments, the biological sample is derived from bodily fluids, e.g., blood, serum, plasma, urine, saliva, stool, breast milk, tears, sweat, cerebral spinal fluid, synovial fluid, semen, vaginal fluid, ascitic fluid, amniotic fluid or cell culture media. In some embodiments, the biological fluid is blood. The biological sample may include cells or may be cell-free.

In another aspect, the preservative compositions of this disclosure reduce or inhibit cell lysis in biological samples. Thus, in one aspect, the disclosure is directed to a method for preserving cells in a biological sample comprising combining a preservative composition of this disclosure and the biological sample. In some embodiments, the biological sample is a cell or tissue sample. In some embodiments, the biological sample is a cell sample derived from bodily fluids, e.g., blood, serum, plasma, urine, saliva, stool, breast milk, tears, sweat, cerebral spinal fluid, synovial fluid, semen, vaginal fluid, ascitic fluid, amniotic fluid or cell culture media, or a lad-derived or modified cell. In a preferred embodiment, the biological fluid is blood, e.g., whole blood or fractions thereof.

The biological sample can be combined with the preservative composition of the disclosure in a number of ways. For example, the biological sample can be collected into a suitable container followed by the addition of the preservative composition to that container, e.g., by syringe or pipette. The preservative composition can alternatively be added to a suitable container for biological sample collection prior to the collection of the biological sample. In some embodiments, the preservative composition is added to a biological sample. In some embodiments, the biological sample is added to the preservative composition.

The disclosure also contemplates methods wherein the components of the preservative composition are added to the biological sample simultaneously or separately. Thus, in some embodiments, this disclosure is directed to methods of preserving nucleic acids and/or cells in a biological sample comprising contacting a biological sample with, in any order or simultaneously, one or more osmotic agents, one or more enzyme inhibitors, optionally one or more metabolic agents, optionally a plasma expander, and one or more of an agent selected from a hydroxyethylstarch, a polymer of N-vinylpyrollidone (NVP), a Ficoll, a protein colloid, a nonprotein synthetic colloid, ethylene diol, propylene glycol, a water-soluble polymer and carboxymethylcellulose or a salt of any of them to provide a treated biological sample. In some embodiments, a suitable container for the collection of the biological sample already contains one or more of the components of the preservative composition, and the remaining components are added to the biological sample, either sequentially, or simultaneously, with the biological sample being collected. For example, a blood collection tube already containing a suitable enzyme inhibitor (e.g., tartaric acid, or EDTA or its salts, or glucaric acid) may be used to collect the biological sample. Subsequent to the collection of the biological sample, the remaining components (i.e., the osmotic agent, optionally one or more of a metabolic inhibitor, an optional plasma expander, and the one or more agents) may be added to the biological sample. In another embodiment, the components of the preservative composition are added to the biological sample, either sequentially, or simultaneously, after the biological sample has been collected. In some embodiments, all of the required components of the preservative composition, and optionally the optional components, are present in the container before the container is used to collect the sample.

In some embodiments, the container to be used for sample collection contains the preservative composition in a lyophilized powder form. In some embodiments, the container to be used for sample collection contains the preservative composition in a granulate form. In some embodiments, the container to be used for sample collection contains the preservative composition in tablet form. In some embodiments, the container to be used for sample collection contains the preservative composition and a suitable vehicle. In some embodiments, the container to be used for sample collection contains the preservative composition as an aqueous solution. In another embodiment, the container to be used is for blood sample collection further comprises an anticoagulant. Examples of anticoagulants include, but are not limited to EDTA (which may also function as an enzyme inhibitor), sodium citrate, citrate-theophylline-adenosine-dipyridamole (CTAD), lithium heparin, sodium heparin, sodium fluoride, acid-citrate-dextrode (ACD), and sodium polyanethol sulfonate. In some embodiments, the suitable container is an evacuated blood sample collection tube.

The amount of the preservative composition that may be combined with a biological sample can be determined by those skilled in the art through routine experimentation. In some embodiments, the ratio of the preservative composition to the biological sample may be from about 1:10 to about 1:1 v/v. In some embodiments, the ratio of the preservative composition to the biological sample is from about 1:8 to about 1:2 v/v. In some embodiments, the ratio of the preservative composition to the biological sample is from about 1:6 to about 1:3 v/v. In some embodiments, the ratio of the preservative composition to the biological sample is from about 1:5 to about 1:4 v/v.

After the biological samples have been stored, the nucleic acids and/or cells may be isolated from the biological sample for analysis using methods known to those skilled in the art including extraction, centrifuge and chromatography methods. Those skilled in the art will recognize that there are many methods that can be used to isolate the nucleic acids and/or cells from a biological sample.

Nucleic acids and/or cells that are preserved using the preservative composition of this disclosure can be isolated from treated biological samples after extended periods of storage under a variety of temperature conditions. In some embodiments, the biological sample that has been contacted with the preservative composition of this disclosure can be stored, either under ambient conditions, or low temperature for at least 1 day, at least 1 week, at least 2 weeks, at least 3 weeks or at least 4 weeks. In some embodiments, the compositions of the disclosure allow for the preservation of a biological sample (i.e., nucleic acids and/or cells in the biological sample) for extended periods of time at a temperature ranging from about −20° C. to about 30° C. In some embodiments, the preservative composition is capable of preserving a biological sample (i.e., nucleic acids and/or cells in the biological sample) for at least 1 week, at least 2 weeks, at least 3 weeks or at least 4 weeks at ambient temperature. In some embodiments the preservative composition is capable of preserving a biological sample for at least 2 weeks at ambient temperature. In some embodiments, the preservative composition of the disclosure is capable of preserving a biological sample (i.e., nucleic acids and/or cells in the biological sample) for at least 1 week, at least 2 weeks, at least 3 weeks or at least 4 weeks at 4° C. In some embodiments, the preservative composition of the disclosure is capable of preserving a biological sample (i.e., nucleic acids and/or cells in the biological sample) for at least 1 week, at least 2 weeks, at least 3 weeks or at least 4 weeks at −20° C.

Nucleic acids (RNA and DNA) that are preserved using the compositions and methods of this disclosure display good yields, purity, integrity and for the RNA amplifiability.

Kits for Providing a Preservative Composition of this Disclosure

The preservative compositions according to the disclosure may be provided as part of a kit that is to be received by the user. The kit allows the preservative composition(s) of this disclosure to be readily combined with a biological sample such that the nucleic acids and/or the cells present in that biological sample are preserved for an extended period of time, e.g., at least 1 week, at least 2 weeks, at least 3 weeks or at least 4 weeks. The preservative composition can be provided such that it is combined with a biological sample after that biological sample has been collected. Alternatively, the preservative composition is provided such that it is combined with the biological sample at the time the biological sample is collected.

In one embodiment, the disclosure is directed to a kit for preserving nucleic acids and/or cells in a biological sample. In some embodiments, the kit comprises a preservative composition as described in the disclosure; and optionally, instructions for use of said preservative composition.

In some embodiments, the preservative composition is provided as an aqueous solution in a dispensing means. In some embodiments the dispensing means is a syringe. The amount of preservative in the dispensing means may be a predetermined amount such that the ratio of the preservative composition that is combined with the biological sample is capable of preserving the nucleic acids and/or cells of that sample over an extended period of time. The kit may further comprise a needle attachable to said syringe. In some embodiments, the kit is for preserving nucleic acids and/or cells in a blood sample, and further comprises a blood collection tube optionally containing a predetermined amount of an anticoagulant.

In some embodiments, the preservative composition is provided in a sealed ampule, wherein said ampule comprises a removable closure, and wherein said ampule is configured to receive a dispensing means upon removal of the closure by the user. In some embodiments, the dispensing means is a pipette or a syringe. In some embodiments, the kit is for preserving nucleic acids and/or cells in a blood sample and further comprises a blood collection tube containing a predetermined amount of an anticoagulant.

In additional embodiments, the kit is directed to preserving nucleic acids and/or cells in a blood sample and comprising a blood collection tube, optionally containing a predetermined amount of an anticoagulant, and a preservative composition of this disclosure.

### Equivalents

The foregoing description and following examples detail certain specific embodiments of the disclosure and describe the best mode of practicing this disclosure as contemplated by the inventors. It will be appreciated, however, that no matter how detailed the foregoing may appear in text, the disclosure may be practiced in many ways and the disclosure should be construed in accordance with the appended embodiments and any equivalents thereof.

Although this disclosure has been described with reference to various applications, methods, compounds, and compositions, it will be appreciated that various changes and modifications can be made without departing from the disclosure herein. The following examples are provided to better illustrate this disclosure and are not intended to limit the scope of the teachings presented herein. While the present disclosure has been described in terms of these exemplary embodiments, the skilled artisan will readily understand that numerous variations and modifications of these exemplary embodiments are possible without undue experimentation. All such variations and modifications are within the scope of the current disclosure.

### EXAMPLES

### Example 1

### Preservation of RNA at Ambient Temperature

Blood samples from two donors are collected into six blood sample collection tubes to assess the ability of a preservative composition according to this disclosure to preserve RNA. The first blood sample from each donor is collected into a blood collection tube containing 2 mL of Composition 1. The second blood sample from each donor is collected into a blood collection tube containing 2 mL of Composition 2. The third blood sample from each donor is collected into a blood collection tube containing 2 mL of Composition 3. The fourth blood sample from each donor is collected into a blood collection tube containing 2 mL of Composition 4. The fifth blood sample from each donor is collected into an EDTA coated blood collection tube. The sixth blood sample from each donor is collected in a plain blood collection tube to serve as control.

Composition 1 (metabolic inhibitor containing, plasma expander free): 25% by weight hydroxyethylstarch, 5% by weight KNO₃, 5% by weight tartaric acid, and 1% by weight thimerosal, and the balance, water.

Composition 2 (metabolic inhibitor free, plasma expander free): 25% by weight hydroxyethylstarch, 5% by weight KNO₃, 5% by weight tartaric acid, and the balance, water.

Composition 3 (metabolic inhibitor containing, plasma expander containing): 25% by weight hydroxyethylstarch, 5% by weight KNO₃, 5% by weight tartaric acid, 1% by weight thimerosal 15% by weight human serum albumin, and the balance, water.

Composition 4 (metabolic inhibitor free, plasma expander containing): 25% by weight hydroxyethylstarch 5% by weight KNO₃, 5% by weight tartaric acid, 15% by weight human serum albumin, and the balance, water.

The samples are stored at room temperature for 28 days. RNA is isolated from all four samples immediately, and after 7 days, 14 days, 21 days and 28 days of storage at room temperature. The RNA is isolated from the samples using extraction and separation techniques known in the art.

The integrity of the RNA from the four samples is analyzed using agarose gel electrophoresis. The bands are compared, and demonstrate that the RNA from the preserved blood samples (samples 1-4) is preserved after 7 days, 14 days, 21 days and 28 days of storage at room temperature, and that the RNA from the samples 5 and 6 is degraded over the same timeframe.

### Example 2

### Preservation of Cell Free DNA

Blood samples from two donors are collected into six blood sample collection tubes to assess the ability of a preservative composition according to the disclosure to preserve cf DNA. The first blood sample from each donor is collected into a blood collection tube containing 2 mL of Composition 1. The second blood sample from each donor is collected into a blood collection tube containing 2 mL of Composition 2. The third blood sample from each donor is collected into a blood collection tube containing 2 mL of Composition 3. The fourth blood sample from each donor is collected into a blood collection tube containing 2 mL of Composition 4. The fifth blood sample from each donor is collected into an EDTA coated blood collection tube. The sixth blood sample from each donor is collected in a plain blood collection tube to serve as control.

Composition 1 (metabolic inhibitor containing, plasma expander free): 25% by weight hydroxyethylstarch, 5% by weight KNO₃, 5% by weight tartaric acid, and 1% by weight thimerosal, and the balance, water.

Composition 2 (metabolic inhibitor free, plasma expander free): 25% by weight hydroxyethylstarch, 5% by weight KNO₃, 5% by weight tartaric acid, and the balance, water.

Composition 3 (metabolic inhibitor containing, plasma expander containing): 25% by weight hydroxyethylstarch, 5% by weight KNO₃, 5% by weight tartaric acid, 1% by weight thimerosal 15% by weight human serum albumin, and the balance, water.

Composition 4 (metabolic inhibitor free, plasma expander containing): 25% by weight hydroxyethylstarch 5% by weight KNO₃, 5% by weight tartaric acid, 15% by weight human serum albumin, and the balance, water.

Each sample is processed immediately, and after 7 days, 14 days, 21 days and 28 days of storage. The plasma from each sample is separated by centrifuge and then transferred to a new tube. The sample DNA is then isolated using techniques known to those skilled in the art.

The DNA is analyzed using Real-Time PCR amplification of Alu 247 (fragments of 247 bp) and Alul 15 (fragments of 115 bp) gene targets. Alu 247 is used to detect the presence of cellular DNA, and Alu 15 is used to detect cell free DNA. An increase of Alu 247 indicates that cell lysis has occurred, resulting in a contamination of the extracellular DNA with cellular DNA. In the event that cell lysis occurs, the observed cycle threshold values (Ct) for the Alu 247 gene target decreases.

The cycle threshold (Ct) values generated from the Real-Time PCR experiments is evaluated, and demonstrates that the preservative is capable of preserving the biological sample for up to 28 days of storage at ambient temperature, and that cell lysis is reduced (samples 1-4). By contrast, the samples lacking the preservative demonstrates decreasing Ct values over time, indicating that cell lysis is occurring more rapidly, resulting in a contamination of the cell free DNA with cellular DNA (samples 5 and 6).

### Example 3

### Determination of Cell Lysis in Treated vs Untreated Biological Samples

Blood samples from two donors are collected into six blood sample collection tubes to assess the ability of a preservative composition according to the disclosure to preserve RNA. The first blood sample from each donor is collected into a blood collection tube containing 2 mL of Composition 1. The second blood sample from each donor is collected into a blood collection tube containing 2 mL of Composition 2. The third blood sample from each donor is collected into a blood collection tube containing 2 mL of Composition 3. The fourth blood sample from each donor is collected into a blood collection tube containing 2 mL of Composition 4. The fifth blood sample from each donor is collected into an EDTA coated blood collection tube. The sixth blood sample from each donor is collected in a plain blood collection tube to serve as control.

Composition 1 (metabolic inhibitor containing, plasma expander free): 25% by weight hydroxyethylstarch, 5% by weight KNO₃, 5% by weight tartaric acid, and 1% by weight thimerosal, and the balance, water.

Composition 2 (metabolic inhibitor free, plasma expander free): 25% by weight hydroxyethylstarch, 5% by weight KNO₃, 5% by weight tartaric acid, and the balance, water.

Composition 3 (metabolic inhibitor containing, plasma expander containing): 25% by weight hydroxyethylstarch, 5% by weight KNO₃, 5% by weight tartaric acid, 1% by weight thimerosal, 15% by weight human serum albumin, and the balance, water.

Composition 4 (metabolic inhibitor free, plasma expander containing): 25% by weight hydroxyethylstarch 5% by weight KNO₃, 5% by weight tartaric acid, 15% by weight human serum albumin, and the balance, water.

Each sample is processed immediately, and after 7 days, 14 days, 21 days and 28 days of storage. The plasma from each sample is separated by centrifuge and is then transferred to a new tube.

Cell lysis is determined by measuring the absorption of free hemoglobin in the isolated plasma (absorption at 414 nm). The samples treated with Compositions 1-4 show low hemoglobin absorption values, indicating that the preservative compositions are preventing cell lysis. By contrast, the sample containing only EDTA and the control sample demonstrate increasing hemoglobin absorption over time, indicating the occurrence of cell lysis.

### Example 4

### Other Preservative Compositions

The experiments described above in Examples 1-3 are repeated with the following preservative compositions. The ability of these composition to preserve the DNA and RNA in the biological samples is generally similar to that of the compositions of Examples 1-3

30% by weight hydroxyethylstarch, 20% by weight sorbitol, 0.5% by weight chlorohexidine, 2% by weight glucaric acid, 15% by weight by weight human serum albumin, the balance being water.

10% by weight Ficoll 400, 20% by weight mannitol, 1% by weight Proclin, 5% by weight tartaric acid, the balance being water.

25% by weight ovalbumin, 10% by weight KNO₃ solution, 7% by weight tartaric acid, 30% by weight gelofusine, the balance being water.

20% by weight propylene glycol, 7% by weight lysine, 2% by weight glucaric acid, 2% by weight thimerosal, the balance being water.

45% by weight dextran, 3% by weight arginine, 15% by weight tartaric acid, 2% by weight thimerosal, the balance being water.

15% by weight polyvinyl pyrrolidone, 15% by weight glycerol, 10% by weight glucaric acid, 35% by weight human serum albumin, the balance being water.

35% by weight polyethylene glycol, 2% by weight KNO₃, 20% by weight tartaric acid, 5% by weight chlorohexidine, the balance being water.

25% by weight ethylene diol, 5% by weight mannitol, 5% by weight glucaric acid, 10% by weight tartaric acid, 5% by weight haemaccel, the balance being water.

20% by weight gelofusine, 5% by weight arginine, 7% by weight glucaric acid, the balance being water.

30% by weight xanthum gum, 7% by weight glycerol, 30% by weight glucaric acid, 0.25% by weight thimerosal, the balance being water.

### Example 5

### Other Embodiments of Preservative Compositions

Other embodiments of the preservative compositions of this disclosure were prepared.

Composition 5 (metabolic inhibitor containing, plasma expander containing): 26.2% by weight PEG 8000, 5.72% by weight NaCl, 0.02% by weight NaN₃, 2.44% by weight 3Na-EDTA, 0.67% by weight Glycerol, and the balance water.

Composition 6 (metabolic inhibitor containing, plasma expander containing): 26.2% by weight PEG 8000, 5.72% by weight NaCl, 0.02% by weight NaN₃, 1.24% by weight 2Na-EDTA, 1.26% by weight 4Na-EDTA, 0.67% by weight Glycerol, and the balance water.

Composition 7 (metabolic inhibitor containing, plasma expander containing): 15% by weight Dextran 40, 5.72% by weight NaCl, 0.02% by weight NaN₃, 1.5% by weight 3K-EDTA, 0.67% by weight Glycerol, and the balance water.

Composition 8 (metabolic inhibitor containing, plasma expander containing): 15% by weight Dextran 70, 5.72% by weight NaCl, 0.02% by weight NaN₃, 1.5% by weight 3K-EDTA, 0.67% by weight Glycerol, and the balance water.

Composition 9 (metabolic inhibitor containing, plasma expander containing): 15% by weight PVP10, 5.72% by weight NaCl, 0.02% by weight NaN₃, 2.5% by weight 3K-EDTA, 0.67% by weight Glycerol, and the balance water.

Composition 10 (metabolic inhibitor containing, plasma expander containing): 15% by weight PVP40, 5.72% by weight NaCl, 0.02% by weight NaN₃, 2.5% by weight 3K-EDTA, 0.67% by weight Glycerol, and the balance water.

Composition 11 (metabolic inhibitor containing, plasma expander containing): 10% by weight Ficoll PM400, 5.72% by weight NaCl, 0.02% by weight NaN₃, 1.24% by weight 2Na-EDTA, 1.26% by weight 4Na-EDTA, 0.67% by weight Glycerol, and the balance water.

Composition 12 (metabolic inhibitor containing, plasma expander containing): 10% by weight Ficoll PM400, 5.72% by weight NaCl, 0.02% by weight NaN₃, 1.5% by weight 3K-EDTA, 0.67% by weight Glycerol, and the balance water.

Composition 13 (metabolic inhibitor containing, plasma expander containing): 26.2% by weight PEG 8000, 0.7% by weight NaCl, 0.02% by weight NaN₃, 1.24% by weight 2Na-EDTA, 1.26% by weight 4Na-EDTA, 0.67% by weight Glycerol, and the balance water.

Composition 14 (metabolic inhibitor containing, plasma expander containing): 2% by weight 1-Vinyl-2-pyrrolidinone (NVP), 0.05% by weight Phenylboronic acid, 5.72% by weight NaCl, 0.02% by weight NaN₃, 1.24% by weight 2Na-EDTA, 1.26% by weight 4Na-EDTA, 0.67% by weight Glycerol, and the balance water.

Composition 15 (metabolic inhibitor containing, plasma expander containing): 2% by weight Carboxymethylcellulose, 5.72% by weight NaCl, 0.02% by weight NaN₃, 1.24% by weight 2Na-EDTA, 1.26% by weight 4Na-EDTA, 0.67% by weight Glycerol, and the balance water.

Composition 16 (metabolic inhibitor containing, plasma expander containing): 10% by weight Ficoll PM400, 5.72% by weight NaCl, 0.02% by weight NaN₃, 1.24% by weight 2Na-EDTA, 1.26% by weight 4Na-EDTA, 0.67% by weight Glycerol, 0.5% by weight D-Saccharic acid Potassium salt, 0.001% by weight Spermine (99 mM stock solution), 0.0007% by weight Spermidine (138 mM stock solution), 0.0008% by weight Putrescine dihydrochloride (124 mM stock solution), and the balance water.

### Example 6

### Analysis of Isolated gDNA and RNA

Blood samples from various donors are collected into the blood sample collection tubes to assess the ability of other embodiments of preservative compositions according to this disclosure to preserve DNA and RNA. Various of the preservative compositions of Example 5 are tested by adding the blood sample into a tube containing 2 mL of the preservative composition.

The gDNA and RNA is isolated from the samples using extraction and separation techniques known in the art. One such gDNA extraction method involves using an Omega BioTeck's Mag-Bind® Blood&Tissue DNA HDQ 96 Kit. One such RNA extraction method involves using a procedure based on Beckman Coulter's RNAdvance Blood Kit.

The isolated gDNA and RNA is analyzed at Day 1, the blood being drawn on Day 0. Quantity, purity and integrity of the nucleic acids as well as the amplifiability of the RNA are analyzed to assess the characteristics of the preservative compositions.

The quantity or the yield of the isolated nucleic acid(s) is identified using a Qubit Broad Range DNA assay and/or RNA assay.

The purity of the nucleic acids is measured by Nanodrop readings of A260/A280 and A260/A230.When A/260/A280 is ˜1.8 and A260/A230 >2, the DNA is considered pure. When A/260/A280 is ˜2 and A260/A230 >1, the RNA is considered pure.

gDNA Integrity

The integrity of the gDNA is analyzed by qPCR of long and short DNA fragments and characterized by the ratio of long fragment over short fragment. When the ratio of is >0.8, DNA is considered intact.

A 10-fold dilution series of the gDNA is prepared for a standard curve. The concentration of gDNA samples is diluted to 10 ng/μL. A forward and reverse primer mix is prepared at 5 μM concentration by mixing 5 μL of 100 μM forward primer, 5 μL of 100 μM reverse primer with 90 μL of nuclease-free water.

Two separate mixtures are prepared with 1 μL of standard or DNA sample, respectively, and 8 μL of nuclease-free water for 1 reaction in mixture #1 and 1 μL of primer mixture and 10 μL of 2× qPCR SYBR Green Master Mix (ThermoFisher Scientific) for 1 reaction in mixture #2. 9μL of mixture #1 and 11 μL of mixture #2 are added to the wells of a 96-well optical plate to run the real-time PCR. The thermal cycling conditions are set forth in the table below.

DNA Thermal Profile

| | | | | |
|---|---|---|---|---|
| Cycle 1 | 95° C. | 10 | min | 1X |
| Cycle 2 | 95° C. | 15 | s | 40X |
| | 64° C. | 30 | s | |
| | 72° C. | 30 | s | |

| | |
|---|---|
| Melting analysis | 1X |

### RNA Integrity

The integrity of RNA from the samples is analyzed by BioAnalyzer using agarose gel electrophoresis and characterized by RNA integrity number (RIN). RIN is an objective metric of total RNA quality ranging from 10 (highly intact RNA) to 1 (completely degraded RNA). Prior to loading the sample onto the BioAnalyzer, RNA samples are diluted to 30 ng/μL, (calculated based on Qubit Quant).

### RNA Amplifiability

The amplifiability of RNA from the samples is analyzed by RT-qPCR.

A serial dilution of control total RNA with known concentration is prepared for the standard curve. The concentration of RNA samples is diluted to 5 ng/μL. The RNA samples are converted into cDNA by reverse-transcription (RT). A master mix of SSIV VILO Master Mix and water is prepared for all samples and standards. 18 μL of Master Mix and 2 μL of standard or diluted RNA samples are added to the wells of a 96-well optical plate or RNase-free tubes. The plate or tubes are flicked, spinned and centrifuged before being placed in a thermal cycler to run the following conditions to obtain cDNA: 25° C., 10 min/50° C., 10 min/85° C., 5 min/4° C., hold.

BRCA1 and ACTB 20X TaqMan Gene Expression Assays are performed by adding 5.5 μL of Master Mix and 4.5 μL of the cDNA to the wells of a 96-well optical plate for real time PCR. The thermal cycling conditions are set in the table below.

RNA Thermal Profile

| | | | | |
|---|---|---|---|---|
| Cycle 1 | 50° C. | 2 | min | 1X |
| Cycle 2 | 95° C. | 2 | min | 1X |

| | | | |
|---|---|---|---|
| Cycle 3 | 95° C. | 1 s or 3 s | 40X |

| | | |
|---|---|---|
| 60° C. | 20 | s |

The Properties of the gDNA in Blood Samples Preserved Using the Compositions of Example 5

The yield of gDNA extracted from blood samples that are preserved in tubes containing various of the individual compositions 5 to 16 is observed to be in a range of 20 to 60 μg/mL(blood and PBS). Taking into account the known variability of blood samples collected from different donors, this is generally a good recovery.

The purity of gDNA extracted from blood samples that are preserved in tubes containing various of the individual compositions 5 to 16 is generally high, as most A260/A280 ratios are observed to be in a range of 1.8 to 1.95 and most A260/A230 ratio are observed to be in a range of 2.0 to 2.5.

The integrity of gDNA extracted from blood samples that are preserved in tubes containing various of the individual compositions 5 to 16 is generally high, as the ratio of long fragment over short fragment (222 bp/90 bp) from the qPCR assay is observed to be close to 1.

### The Properties of the RNA in Blood Samples Preserved Using the Compositions of Example 5

The yield of RNA extracted from blood samples that are preserved in tubes containing various of the individual compositions 5 to 16 is observed to be in a range of 2-11 μg/mL (blood and PBS). Taking into account the known variability of blood samples collected from different donors, this is a good recovery.

The purity of the RNA extracted from blood samples that are preserved in tubes containing various of the individual compositions 5 to 16 is generally high, as most A260/A280 ratios are observed to be in a range of 1.75 to 2.35 and most A260/A230 ratios are observed to be in a range of 0.8 to 1.9.

The RIN of the RNA isolated from blood samples that are preserved in tubes containing various of the individual compositions 5 to 16 is generally high, as most samples are observed to have a RIN of >5 and preferably >7.

The amplifiability of the isolated RNA is generally good, the PCR quantity that is observed being in a range of 5-20 ng, preferably 8-20 ng, for ACTB and 0.3-1 ng, preferably 0.6-1 ng, for BRCA 1.

We claim:

1. A preservative composition comprising:
a. one or more of an osmotic agent;
b. one or more of an enzyme inhibitor;
c. optionally one or more of a metabolic inhibitor;
d. optionally a plasma expander; and
e. one or more of an agent selected from a hydroxyethylstarch, a polymer of N-vinylpyrollidone (NVP), a Ficoll, a protein colloid, a non-protein synthetic colloid, ethylene diol, propylene glycol, a water-soluble polymer and carboxymethylcellulose or a salt thereof.

2. The preservative composition according to claim 1, wherein the one or more agents is present in an amount of about 10% to about 50% by weight of the composition.

3. The preservative composition according to claim 1 or 2, wherein the protein colloid is selected from an albumin, an ovalbumin, or a gelatin colloid.

4. The preservative composition according to claim 3, wherein the albumin comprises a human serum albumin (HSA), a bovine serum albumin (BSA) or an ovalbumin.

5. The preservative according to claim 3, wherein the gelatin colloid is a urea-linked gelatin, a succinylated gelatin, or an oxypolygelatin.

6. The preservative composition according to claim 1 or 2, wherein the non-protein colloid is a hydroxyethylated starch (HES) or a dextran.

7. The preservative composition according to claim 1 or 2, wherein the water soluble polymer is a polyacrylamide, a polyacrylate, a polydextrose, a polyglycine, a polyethyleneimine, a polylysine, a polyethylene glycol, a polyvinyl pyrrolidone, a polyvinyl alcohol, a polyacrylic acid, a N-(2-hydroxypropyl) methacrylamide, a divinyl ether-maleic anhydride, a polyoxazoline, a polyphosphate, a polyphosphazene, a xanthan gum, a pectin, a chitosan derivative, a dextran, a carrageenan, a guar gum, a cellulose ether, a sodium carboxymethyl cellulose, a hydroxypropyl cellulose, a hypromellose, a hyaluronic acid, an albumin, a starch, or a starch based derivative.

8. The preservative composition according to any one of claims 1-7, wherein the one or more osmotic agents is a hypertonic or isotonic salt solution, including, but not limited to sodium, potassium, magnesium and calcium containing solutions, Ringer's lactate, Ringer's acetate, an amino acid, sorbitol, glycerol, mannitol, sugars such as sucrose or glucose, tartaric acid, and glucaric acid or salts of any of them.

9. The preservative composition according to claim 8, wherein the osmotic agent is present in an amount of about 0.5% to about 10% by weight of the composition.

10. The preservative composition according to claim 9, wherein the osmotic agent is present in an amount of about 1% to about 20% by weight of the composition.

11. The preservative composition according to claim 9, wherein the osmotic agent is present in an amount of about 0.5% to about 10% by weight of the composition.

12. The preservative composition according to any one of claims 1-11, wherein the one or more enzyme inhibitor is ethylenediaminetetraacetic acid (EDTA), hydroxyethylethylenediaminetriacetic acid (HEDTA), dithiothreitol (DTT), ethylene glycol-bis(β-aminoethyl ether)-N,N,N′,N′-tetraacetic acid (EGTA), citric acid, oxalate, aurintricarboxylic acid (ATA), tartaric acid, glucaric acid, or salts of any of them, including but not limited to sodium and potassium salts.

13. The preservative composition according to claim 12, wherein the enzyme inhibitor is present in an amount of about 0.5% to about 30% by weight of the composition.

14. The preservative composition according to claim 13, wherein the enzyme inhibitor is present in an amount of about 1% to about 30% by weight of the composition.

15. The preservative composition according to claim 13, wherein the enzyme inhibitor is present in an amount of about 0.5% to about 5% by weight of the composition.

16. The preservative composition according to any one of claim 1-15 wherein the one or more optional metabolic inhibitor is sodium azide, thimerosal, proclin, or chlorohexidine.

17. The preservative composition according to claim 16, wherein the metabolic inhibitor is present in an amount of about 0.01% to about 10% by weight of the composition.

18. The preservative composition according to claim 17, wherein the metabolic inhibitor is present in an amount of about 0.01% to about 5% by weight of the composition.

19. The preservative composition according to any one of claims 1-18, wherein the optional plasma expander is glycerol, a protein colloid (e.g., albumin, ovalbumin, and gelatins) or a non-protein colloid (e.g., hydroxyethyl starch).

20. The preservation composition according to claim 19, wherein the plasma expander is present in an amount of about 0.5% to about 40% by weight of the composition.

21. The preservation composition according to claim 20, wherein the plasma expander is present in an amount of about 1% to about 40% by weight of the composition.

22. The preservative composition according to claim 19, wherein:
a. the protein colloid is albumin, ovalbumin or a gelatin; and
b. the non-protein colloid is hydroxyethyl starch.

23. The preservative composition according to any one of claims 1-22, that is in the form of a lyophilized dry powder.

24. The preservative composition according to any one of claims 1-22, that is in the form of an aqueous solution.

25. The preservative composition according to any one of claims 1-24, that further comprises a biological sample.

26. The preservative composition according to claim 25, wherein the biological sample is derived from a bodily fluid.

27. The preservative composition according to claim 26, wherein the bodily fluid is blood, plasma, serum, urine, saliva, stool, breast milk, tears, sweat, cerebral spinal fluid, synovial fluid, semen, vaginal fluid, ascitic fluid, amniotic fluid or cell culture media.

28. The preservative composition according to claim 27, wherein the bodily fluid is whole blood or fractions thereof.

29. The preservative composition according to claim 26, wherein the biological sample is a lab-derived or modified cell.

30. The preservative composition according to any one of claims 25-29, wherein the biological sample comprises a nucleic acid selected from the group of RNA, DNA or a combination thereof.

31. The preservative composition according to claim 30, wherein the nucleic acid is cell-free RNA, cell-free DNA or a combination thereof.

32. The preservative composition according to claim 30, wherein the nucleic acid is cellular RNA, cellular DNA or a combination thereof.

33. The preservative composition according to any one of claims 25-32, wherein the ratio of the preservative composition to the biological sample is from about 1:10 to about 1:1 v/v.

34. The preservative composition according to claim 33, wherein the ratio of the preservative composition to the biological sample is from about 1:5 to about 1:4 v/v.

35. The preservative composition according to any one of claims 1-34, wherein the composition is capable of preserving the nucleic acids and/or cells in a biological sample for at least 2 weeks at ambient temperature.

36. A method for preserving nucleic acids and/or cells in a biological sample comprising the steps combining a preservative composition according to any one of claims 1-24, and the biological sample.

37. The method according to claim 36, wherein the biological sample is derived from a bodily fluid.

38. The method according to claim 37, wherein the bodily fluid is blood, plasma, serum, urine, saliva, stool, breast milk, tears, sweat, cerebral spinal fluid, synovial fluid, semen, vaginal fluid, ascitic fluid, or amniotic fluid.

39. The method according to claim 38, wherein the bodily fluid is whole blood or fractions thereof.

40. The method according to any one of claims 36-39, wherein the cells are stem cells, bone cells, blood cells, muscle cells, fat cells, skin cells, nerve cells, endothelial cells, sex cells, pancreatic cells, cancer cells, tumor cells, circulating tumor cells or lab-derived or modified cells.

41. The method according to any one of claims 36-40, wherein the nucleic acid is RNA, DNA or a combination thereof.

42. The method according to claim 41, wherein the nucleic acid is cell-free RNA, cell-free DNA or a combination thereof.

43. The method according to claim 41, wherein the nucleic acid is cellular RNA, cellular DNA or a combination thereof.

44. The method according to any one of claims 36-43, wherein the ratio of the preservative composition to the biological sample is from about 1:10 to about 1:1 v/v.

45. The method according to any one of claims 36-44, wherein the ratio of the preservative composition to the biological sample is from about 1:5 to about 1:4 v/v.

46. A kit for preserving nucleic acids and/or cells in a biological sample comprising:
a. a preservative composition according to any one of claims 1-24; and
b. optionally, instructions for use of said preservative composition.

47. The kit according to claim 46, wherein the biological sample is derived from a bodily fluid.

48. The kit according to claim 47, wherein the bodily fluid is blood, plasma, serum, urine, saliva, stool, breast milk, tears, sweat, cerebral spinal fluid, synovial fluid, semen, vaginal fluid, ascitic fluid, or amniotic fluid.

49. A kit for preserving nucleic acids and/or cells in a blood sample comprising:
a. a blood collection tube containing a predetermined amount of an optional anticoagulant;
b. a syringe containing a predetermined amount of a preservative composition according to any one of claim 1-22 or 24; and
c. optionally, a needle attachable to said syringe.

50. A kit for preserving nucleic acids and/or cells in a blood sample comprising:
a. a blood collection tube optionally containing a predetermined amount of an anticoagulant; and
b. a sealed ampule, containing a predetermined amount of a preservative composition according to any one of claims 1-24, wherein said ampule comprises a removable closure and wherein said ampule is configured to receive a dispensing means upon removal of the closure by a user.

51. A kit for preserving nucleic acids and/or cells in a blood sample comprising a blood collection tube containing a predetermined amount of an optional anticoagulant and a preservative composition according to any one of claims 1-24.

52. The kit according to any one of claims 46-51, wherein the nucleic acid is RNA, DNA or a combination thereof.

53. The kit according to claim 52, wherein the nucleic acid is cell-free RNA, cell-free DNA or a combination thereof.

54. The kit according to claim 52, wherein the nucleic acid is cellular RNA, cellular DNA or a combination thereof.

55. A method for preserving cells in a biological sample comprising the steps of combining a preservative composition according to any one of claims 1-24 and the biological sample.
